(19) United States
(12) Patent Application Publication
(10) Pub. No.: US 2008/0305129 A1
(43) Pub. Date: Dec. 11, 2008

(54) Highly Attenuated Pox Virus Strains, Method for the Production Thereof and the Use Thereof as Paramunity Inducers or For Producing Vector Vaccines

(76) Inventor: Anton Mayr, Starnberg (DE)

Correspondence Address: MCDONNELL BOEHNEN HULBERT & BERGHOFF LLP, 300 S. WACKER DRIVE, 32ND FLOOR, CHICAGO, IL, 60606, US

(21) Appl. No.: 11/917,319

(22) PCT Filed: Jun. 16, 2006
(86) PCT No.: PCT/EP06/05781

§ 371 Date: Aug. 7, 2008

(30) Foreign Application Priority Data
Jun. 16, 2005 (DE) 102005027956.2-41

(51) Int. Cl. A61K 48/00; C12N 7/04; A61K 39/275

(52) U.S. Cl. 424/205.1; 435/236; 424/93.2

(57) ABSTRACT

The present invention relates to highly attenuated animal smallpox viral strains and to the use thereof as paramunity inducers or for producing vector vaccines. As a result of the high attenuation process, the claimed animal smallpox strains lose their virulent and immunising properties. The invention also relates to a method for producing such highly attenuated pox virus strains and the use thereof for inducing paramunity, i.e. for activating the non-specific immune system in mammals and humans or for producing vector vaccines for specific immunisation with the positive side-effect of paramunisation. The claimed highly attenuated animal smallpox viruses are thus suitable for preventing and treating diseases associated with an immune deficiency. Preferred embodiments relate to highly attenuated orthopox—(e.g. camel smallpox viruses), leporipox—(e.g. myxoma viruses), avipox-, parapox- and other orthopox viral strains, such as MVA, which have excellent paramunisation properties and in which the immunising properties have been lost.

### BACKGROUND OF THE INVENTION

The present invention relates to highly attenuated animal poxviruses, to paramunity inducers produced therefrom and to vector vaccines based on highly attenuated animal poxvirus strains. The highly attenuated animal poxviruses of the invention possess, owing to the highly attenuating process, no virulent and immunizing properties whatsoever. A further aspect of the invention relates to methods for producing such highly attenuated animal poxvirus strains and to the use thereof as paramunity inducers for inducing paramunity, i.e. for activating the nonspecific (paraspecific) immune system in humans and animals or as vector vaccine for immunizing a mammal or human. The highly attenuated animal poxviruses of the invention are further suitable for the prophylaxis and treatment of multifactorial, usually chronic disorders. Preferred embodiments of the invention relate to highly attenuated animal poxvirus strains of all genera of the family poxviridae, which strains have been isolated from infected animals and highly attenuated by serial passages. The animal pox strains of the invention have excellent paramunizing properties, the virulent and immunizing properties having been lost owing to the highly attenuating method of the invention.

The endogenous immune system of mammals can be divided into an antigen-specific and an antigen-nonspecific (paraspecific) part. The antigen-specific part of the immune system includes for example antibodies or specific immune cells. The antigen-specific mechanisms are responsible for establishing a specific immunity, while the antigen-nonspecific are responsible for building up paramunity. The paraspecific activities of the antigen-nonspecific immune system (or “innate immune system”) include nonselective cellular and soluble protective elements such as, for example, the complement lysozyme system and the regulatory cytokine cascade, and cellular protective elements such as, for example, granulocytes, micro- and macrophages, natural killer cells, non-antigen-conditioned T lymphocytes, dendritic cells and others.

Paramunity means the state of a well-regulated and optimally functioning nonspecific defense system which confers on the organism a rapidly developing, time-limited, increased protection from a large number of different pathogens, antigens and other noxae.

Paraspecific activities are to be detected in the relevant organism immediately after contact with noxae, i.e. endogenous or exogenous harmful substances, and transformed endogenous cells, after about 2 to 6 hours, while the effects of the antigen-specific immune system appear only after 5-8 days (cellular specific immunity) or even after weeks (antibodies). Additional time is gained in this way in order to build up specific defense reactions against the antigens which could not be neutralized by the paramunizing activities. The paraspecific defense therefore makes it possible for the organism to defend itself immediately, i.e. without loss of time, on confrontation with a wide variety of foreign materials, infectious pathogens, toxins and transformed endogenous cells (Anton Mayr, “Paramunisierung: Empirie oder Wissenschaft”, Biol. Med., edition 26(6): 256-261, 1997).

The paraspecific immune defense is thus a physiological process and can be defined as “primary barrier” in a confrontation with a harmful substance-containing environment. This form of defense is irreplaceable not only for the lower organisms, but in particular also for the more highly developed and highly developed life forms. Thus, it emerges that primary congenital defects in this biological defense system may lead to life-threatening situations. An example which is to be mentioned is the “Chediak-Steinbrinck-Higashi syndrome” in humans, which is characterized by granulocyte defects and dysfunctions of the natural killer cells (NK cells) and in most cases leads to the death of the patient by completion of the 10th year of life.

The condition of paramunity is characterized by an increased rate of phagocytosis, an increased function of the spontaneous cell-mediated cytotoxicity (NK cells) and an increased activity of other non-antigen-specific lymphoreticular cells. At the same time there is release of particular cytokines which have stimulating and/or suppressing effects (e.g. via repressor mechanisms), i.e. have optimal regulatory effects, both with the cellular elements and with one another. This closely linked and stepwise responding biological system of paramunity with its various acceptor, effector and target cells, and the signal-transmitting molecular messengers (cytokines) is moreover thoroughly connected to the hormonal and nervous systems, and in some cases even with the vascular and metabolic systems. It is thus an important component of communication, interaction and regulation of the defense network which is naturally present in every organism from birth onwards. Nature has thus provided all organisms with appropriate protection from the outset. During phylogenesis there was initially development only of the paraspecific, i.e. nonspecific defense system. Only during the later course of evolution did the specific immune system develop stepwise.

Paramunity is induced medically by paramunization with so-called paramunity inducers. Medical paramunization is achieved by activating the cellular elements of the paraspecific part of the immune system and the formation, linked thereto, of cytokines, with the aim of eliminating dysfunctions, rapidly increasing the pathogen- and antigen-nonspecific protection of an individual (optimal bioregulation), eliminating an immunosuppression or immunodeficiency which has arisen as a result of stress or in other ways (e.g. pharmacologically), repairing deficits and/or acting as a regulator between the immune, hormonal and nervous systems (Anton Mayr, “Paramunisierung: Empirie oder Wissenschaft”, Biol. Med., edition 26(6): 256-261, 1997). This means that certain nonspecific endogenous defense processes can be increased, supplemented or else depressed, depending on the type of paramunization and the responsiveness, such as, for example, the patient's defense status.

The paramunity inducer per se is a protein, i.e. it is not comparable either to an antibody or to a chemical, an antibiotic, vitamin or hormone. On the contrary, it activates like a catalyst by a stepwise mechanism the paraspecific immune system so that the latter sufficiently mobilizes cellular and humoral defense mechanisms. Paramunity inducers in this case have both regulatory and repair effects on the immune defenses. Concerning the mode of action of paramunity inducers, it is known that they are taken up by phagocytic cells (acceptor cells) which are thus activated and release mediators such as, for example, cytokines, which in turn mobilize effector cells.

Paramunity inducers based on combinations of two or more conventionally attenuated animal poxvirus components which are derived from different animal poxvirus strains with paramunizing properties are described in European patent EP 0 669 133 B1.

The animal poxvirus strains on which these paramunity inducers are based have been attenuated in a conventional way, i.e. they are in a reduced condition in which the virulent and, in particular, the immunizing properties of the virus have been weakened but not completely lost.

The present invention presents for the first time a novel method which completely eliminates the virulence and immunogenicity of simply attenuated animal poxvirus strains. This is referred to hereinafter as a “highly attenuating” method.

Such a high attenuation of poxviruses is shown in the present invention for the first time on the basis of the orthopoxviruses camelpox virus (Orthopoxvirus cameli) and ektromelia virus (Orthopoxvirus muris), the leporipoxvirus myxomatosis virus (Leporipoxvirus myxomatosis), the avipoxviruses fowlpox virus (Avipoxvirus gallinae) and canarypox virus (Avipoxvirus serinis) and the parapoxvirus ecthyma (Parapoxvirus ovis).

Exemplary embodiments of the invention relate to the high attenuation of the orthopoxvirus strain camelpox virus strain h-M 27 and the leporipoxvirus strain myxomatosis virus strain h-M 2. Neither an attenuation nor a high attenuation has to date been carried out or described for these poxvirus strains. Other preferred embodiments relate to further orthopoxvirus strains, and to strains of parapoxviruses and avipoxviruses (see below).

A simple conventional attenuation has been shown for the genus Orthopoxvirus in the case of the vacciniavirus ankara MVA by A. Mayr, H. Stickl, H. K. Müller, K. Danner and H. Singer, 1978: “Der Pockenimpfstamm MVA”, Zbl. Bakt. Hyg., I. Abt. Orig. B 167, 375-390; Mayr, A., 1999: “Geschichtlicher Überblick über die Menschenpocken (Variola), die Eradikation von Variola und den attenuierten Pockenstamm MVA”, Berl. Münch. TierärztlWschr. 112, 322-328; for the genus avipoxvirus HP1 and KP1 by A. Mayr, F. Hartwig, and I. Bayr, 1965: “Entwicklung eines Impfstoffes gegen die Kanarienpocken auf der Basis eines attenuierten Kanarienpockenkulturvirus”, Zbl. Vet. Med. B 12, 41-49; A. Mayr and K. Malicki, 1966: “Attenuierung von virulentem Hühnerpockenvirus in Zellkulturen und Eigenschaften des attenuierten Virus”, Zbl. Vet. Med. B. 13, 1-13; and for the genus parapoxvirus ORF-1701 by A. Mayr and M. Büttner, 1990: “Ecthyma (ORF) virus”: In: Z. Dinter and B. Morein (eds.): Virus infections of vertebrates, vol. 3; Virus infections of ruminants, Elsevier Science Publishers, B.V. Amsterdam.

Some of the animal poxvirus strains highly attenuated by the method of the invention are explained in more detail below:

### Camelpox Virus

Camelpox are the pathogens of a dangerous viral disease of camelids which has a cyclic systemic course and is characterized by an exanthema preferentially of the skin and mucous membrane in the head, neck and throat region and the extremities and the inguinal region (Munz, E., 1999: “Pox and pox-like diseases in camels”, Proc. 1st Int. Camel Conf. 1, 43-46). The disease occurs cyclically every 2 to 3 years if a sufficiently large sensitive population is available. Two genera (lama and camelus) of the family Camelidae are preferentially affected by camelpox viruses (Mayr, A. and Czerny C. P., 1990: “Camelpox virus”, In: Dinter Z. and Morein B. (eds.): “Virus infections of vertebrates”, vol. 3: Virus infections of ruminants. Elsevier Science Publishers B.V. Amsterdam). The genus Camelus includes the one-humped dromedary (Camelus dromedarius) and two-humped Bactrian camel (Camelus ferus bactrianus). Dromedaries and Bactrian camels occur mainly in the countries of the so-called “old world” (deserts, steppes of north Africa, Arabia, Mongolia), while the preferred habitat of the lama is in south America.

Camelpox virus (Orthopoxvirus cameli) is a particularly close relative of the variola virus, the pathogen of smallpox in humans (Variola). Camelpox virus is not pathogenic for humans. Camelpox virus is, like all classical poxviruses, brick-shaped and has characteristic surface proteins which are responsible for the immunizing and paramunizing properties of the virus or its constituents. The average size is, depending on the genus and strain, 280 nm in the longitudinal direction and about 180 nm in the transverse direction (Otterbein, C. K., 1994: “Phäno-und genotypische Untersuchungen zweier Kamelpoxvirus-Isolate vor und nach Attenuierung durch Zellkulturpassagen”, Vet. Med. Diss. Munich). The genome of the camelpox virus consists of a linear double-stranded DNA. The two DNA strands are covalently bonded together at the genome ends, so that the virus DNA forms a continuous polynucleotide chain.

### Myxomatosis Virus

Myxomaviruses are the pathogens of myxomatosis, a contagious systemic viral disease of wild and domestic rabbits which progresses in cycles and is characterized by generalized, in some cases hemorrhagic subcutaneous edemas on the head and over the entire body, with preference for the anal region, the vulva and the tube, unlike any other infectious disease. Introduction of myxomatosis into a country previously free of the disease results in a rapid and fatal progression. After the virus has become endemic, the character of the disease changes until the infections are clinically inapparent (Mayr A.: Medizinische Mikrobiologie, Infektions-und Seuchenlehre, 7th edition, Enke-Verlag, Stuttgart, 2002).

The disease is widespread among American cottontail rabbits of the genus Sylvilagus which occupy exclusively the new world. These wild rabbits form the only natural reservoir of the disease. The infection takes a mild form in them. By contrast, the disease has an almost 100% mortality in European wild and domestic rabbits of the genus Oryctolagus, which are also naturalized in Australia, when the pathogen is introduced.

The natural host range of the Myxomavirus (genus Leporipoxvirus) has narrow limits. In general, the virus replicates only in American cottontail rabbits and in European domestic and wild rabbits. However, a few infections in European wild hares have also been observed. Attempted transmission to other animal species and to humans had negative results.

### Avipoxvirus

The infections caused by avipoxviruses, especially fowlpox virus and canarypox virus, progress in a similar way. Fowlpox derive from Asia and have been known for thousands of years. They are distributed around the world and very resistant. Transmission takes place by entry through skin wounds. Stinging insects may also be involved in transmission. The incubation time for the disease is 4 to 14 days. There are two forms, a distinction being made between the so-called cutaneous form and the mucosal form. The cutaneous form is characterized by blisters or scabby nodules on the head, comb, neck and feet. The mucosal form exhibits yellowish white deposits on the tongue, the mucous membranes of the beak, of the larynx, of the trachea and the eyes.

The incubation time on infection with canarypox virus is 3 to 16 days. After the disease has broken out, most of the stock dies within only a few hours. The infected birds show nodules on the horny parts and at the angles of the beak. Massive respiratory impairments occur, and the birds rapidly suffocate from the caseous deposits in the airways caused by the virus.

Attenuated strains of avipoxviruses have been obtained by successive passages in chicken embryo fibroblast cell cultures and have been employed for vaccinating chickens. The most investigated and available strain is the strain HP-1 (A. Mayr and K. Malicki, 1966: “Attenuierung von virulentem Hühnerpockenvirus in Zellkulturen und Eigenschaften des attenuierten Virus”, Zbl. Veg. Med. B 13, 1-13). More than 200 passages in chicken embryo fibroblasts lead to an attenuated virus but which is still capable of replication and retains pathogenicity for chickens on intravenous or aerosol administration. Viruses passaged more than 400 times are regarded as apathogenic and are regarded as efficient and extremely safe vectors for use in mammals. It was possible to achieve immunization without complete productive replication of the virus taking place.

### OBJECT OF THE INVENTION

The animal poxvirus strains which have been weakened to date by conventional attenuation lead to an increase in paramunizing properties and to a reduction in the virulent and immunizing properties of the virus and its constituents. However, not all virulent and immunizing properties of the animal pox strains are lost in conventional attenuation. Immune responses are still present in mammals with simply attenuated animal pox strains. This is presumably related to a simple attenuation having too little stability or the attenuation being too low in the case of simply attenuated animal poxviruses.

The present invention is therefore based on the object of providing animal pox strains which are stable, exhibit a high degree of attenuation and in which the poxviruses are modified in such a way that they have completely lost their virulent and immunizing properties and thus can be used as harmless paramunity inducers and vector vaccines.

This object is achieved according to the invention by the subject matters of the appended claims.

It has surprisingly been found that simply attenuated animal pox strains are modified by additional attenuation steps with continuing plaque terminal dilution passages in selected permissive cell cultures in such a way that they completely lose their virulence and immunizing ability without their ability to replicate being impaired. The animal pox strains of the invention are also further restricted in their host range. The deletions resulting from the high attenuation in the viral genome additionally make it possible to introduce foreign antigens.

The loss, caused by the high attenuation, of the immunizing proteins makes additional paramunizing proteins active, and they increase in a significant manner the paramunizing activity of these strains. In this way, highly active and harmless paramunity inducers are obtained and do not cause any allergies or other immunopathogenic side effects even if administrations are repeated in the short term and are frequent.

The animal pox strains which are highly attenuated by the method of the invention are therefore outstandingly suitable as paramunity inducers or for producing vector vaccines.

### SUMMARY OF THE INVENTION

The invention relates to highly attenuated animal poxvirus strains and to the use thereof as paramunity inducers or for producing vector vaccines.

Particular embodiments of the highly attenuated animal pox strains are strains of the myxomatosis virus and of the camelpox virus. Particular preference is given to the camelpox virus strain h-M 27 with the deposit number 05040602 and the myxomatosis virus strain h-M 2 with the deposit number 05040601. The viruses were deposited at the depositary institution of the Public Health Laboratory Service (PHLS), Centre for Applied Microbiology & Research (CAMR), European Collection of Animal Cell Cultures (ECACC), Porton Down, Salisbury, Wiltshire, United Kingdom. Other embodiments of the invention relate to the high attenuation of the canarypox virus (Avipoxvirus serinae), preferably of the strain KP1, of the ectromelia virus (Orthopoxvirus muris), preferably of the strain Mü 1 and of the fowlpox virus (Avipoxvirus gallinae), preferably of the strain HP1 and of the parapox virus (Parapoxvirus ovis).

In the high attenuation of animal poxvirus strains discovered according to the invention, the virulence of the viral strains and their immunizing properties are completely lost by comparison with conventionally attenuated animal pox strains. The highly attenuated animal poxviruses of the invention therefore no longer have any residual virulence or immunogenity. The highly attenuated animal pox strains are therefore particularly suitable for use as paramunity inducers or for producing vector vaccines.

The invention further relates to methods for producing the highly attenuated poxvirus strains of the invention. Preferred poxvirus strains are strains which belong to the genus orthopoxvirus, avipoxvirus, leporipoxvirus and parapoxvirus.

The high attenuation of poxvirus strains is achieved according to the invention by additional plaque terminal dilution passages (i.e. transfer and continuation) of conventionally attenuated viral strains in optimized, selected, permanent cell lines (e.g. VERO cells), in primary cell cultures (e.g. chicken embryo fibroblasts (FHE) cell cultures), incubated chicken eggs or in experimental animals. It has surprisingly been found that the virulence and immunogenicity of the animal poxviruses and their constituents is lost by comparison with conventionally attenuated strains through this additional passaging. The passaging in the selected cell systems or cultures takes place until the desired properties are achieved, i.e. until the animal poxviruses no longer have any virulence or immunogenicity and instead show an increased activity of the nonspecific immune system (paramunity). This can normally be achieved by at least 300-500 passages in optimized cell systems such as cell cultures of VERO cells passages (ATCC CCL-81, WHO, American Type Culture Collection).

Such optimized cell systems provide the necessarily high infectious titers. The further desired biological, genetic and immunological properties resulting from a high attenuation of animal pox strains are listed in table 3.

The method of the invention for producing highly attenuated animal poxviruses can be generally defined by the following steps:
(a) adaptation of the animal pox to a permissive cell system for example consisting of the chorioallantoic membrane of 10-day old chicken embryos (CAM) or cell cultures, e.g. lamb kidney cell cultures;
(b) transfer and continuation of the animal poxviruses for attenuation by long-term passages in various permissive cell systems which make optimal infectious titers possible, especially AVIVER or VERO cells;
(c) transfer and continuation of the animal poxviruses for attenuation in an optimal cell system for about 100-300, e.g. 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295 or 300 passages, preferably in VERO, AVIVER or MA cells, it being preferred for the cell system used in (c) to differ from the cell system used in (b);
(d) transfer and continuation of the animal poxviruses in VERO cells for at least 90, e.g. 95, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300 or more passages, preferably plaque terminal dilution passages.

In a particularly preferred embodiment, the highly attenuated orthopoxvirus strain is a highly attenuated camelpox virus (Orthopoxvirus cameli), especially of the strain h-M 27. A preferred method for highly attenuating camelpox viruses includes the following steps:
(a) culturing the isolated camelpox virus for about 2 to 4 passages in lamb kidney cell cultures;
(b) transfer and culturing of the animal poxviruses for about 5 to 10 passages in VERO cells passages (ATCC CCL-81, WHO, American Type Culture Collection);
(c) transfer and culturing of the animal poxviruses for about 114 to about 150 passages in MA cells;
(d) transfer and culturing of the animal poxviruses for about a further 267 or more, e.g. 270, 275, 280, 285, 290, 295, 300, 305, 310, 315, 320, 325, 330, 335, 340, 345, 350, 355, 360, 365, 370, 375, 380, 385, 390, 395, 400 or more passages in VERO cells, preferably plaque terminal dilution passages.

The animal poxviruses generated in this way can be employed for producing paramunity inducers and vector vaccines. For the production the virus harvest capable of replication is used.

A further embodiment of the present invention relates to the highly attenuated leporipoxvirus strain myxomatosis virus (Leporipoxvirus myxomatosis), strain h-M 2.

A preferred method for highly attenuating such a myxomatosis virus strain, preferably the strain h-M 2, includes the following steps:
(a) isolation from diseased animals via the chorioallantoic membrane of 10-day old chicken embryos (CAM) and continuation in this system for at least 2 or more, e.g. 3, 4, 5, 6, 7, 8, 9, 10 or more passages;
(b) transfer and continuation of the isolated animal poxviruses for at least 120 or more, e.g. 125, 130, 135, 140, 145, 150, 155, 160, 165, 170, 175, 180 or more passages in VERO cell cultures;
(c) transfer and continuation of the animal poxviruses in AVIVER cells for at least 24 or more, e.g. 25, 30, 35, 40, 45, 50, 55, 60 or more passages;
(d) transfer and continuation of the animal poxviruses in VERO cells for at least 157 to 200 passages;
e) transfer and continuation of the animal poxviruses in MA cells for at least 114 to 150 passages;
f) transfer and continuation of the animal poxviruses in VERO cells for at least 179 passages.

The virus harvests are inactivated by treatment with beta-propiolactone to produce paramunity inducers.

The invention also relates to pharmaceutical compositions which comprise one or more highly attenuated pox strains of different origin in combination, and to which a pharmaceutical carrier is added where appropriate.

A further aspect of the invention therefore relates to the use of one or more highly attenuated animal pox strains of the invention (e.g. in combination) or constituents of the highly attenuated animal pox strains for activating the paraspecific immune system in a mammal or in a human for prophylaxis and therapy.

In a further aspect of the present invention, the highly attenuated animal poxviruses are employed to produce vector vaccines; the virus harvests capable of replication are used for this purpose. A nucleic acid coding for a foreign antigen is in this case incorporated into one of the deletions, resulting from the high attenuation, of the nucleic acid of the vector (animal poxvirus) so that the foreign gene can be expressed by the vector. The foreign proteins resulting in this way provide immunizing epitopes and thus stimulate the endogenous specific defense system.

### DEFINITIONS

The term “attenuation” (attenuate: weaken, mitigate) of an infectious pathogen (e.g. viruses, bacteria, fungi) means in principle the reduction of its virulent and immunizing properties. In particular, depending on the degree of attenuation, in gene technology terms there is a reduction in the molecular weight and thus a shortening of its nucleic acid, associated with the occurrence of deletions, in biological terms there is a reduction or loss of its pathogenic properties in relation to virulence and contagiousness, in immunological terms there is a loss of immunogenic activities and an increase in the paraspecific potencies, and in clinical terms there is a restriction in the host range and an increased activity of paraspecific defense reactions of the host.

“High attenuation” means the further reduction of simply attenuated but still partly virulent and immunizing pathogens until the virulence and the immunizing potential are completely lost, the high attenuation resulting in an extreme limitation of the host range. The high attenuation greatly increases the paramunizing potential. In relation to reactivation of their lost virulent and immunizing properties, the highly attenuated poxviruses are more stable than conventionally attenuated strains, i.e. conversion back is impossible.

The highly attenuated animal pox strains differ from the conventionally attenuated animal pox strains in gene technology terms by a further decrease in the molecular weight of the viral nucleic acid and an increase in deletions in the nucleic acid; in biological terms by complete loss of virulence and contagiousness, these strains simultaneously achieving an optimal infectious titer, which is high by comparison with conventionally attenuated strains, in the permissive host system; in immunological terms by total loss of immunogenicity, and in molecular biology terms by loss of cytokine receptors, e.g. receptors for interferon and certain interleukins.

The terms “virulence” and “virulent properties” are used synonymously in the present invention. Virulence refers to the degree of the disease-causing properties of a certain strain of a pathogenic species in a particular host under defined infection conditions. The degree of virulence may vary widely within the strains of a species. A distinction is made between highly, weakly and non-virulent (avirulent) strains. A change in the host and environmental conditions may also lead to a change in the virulence of the strain, but it may also remain unchanged. Thus, the host's defenses, the anatomical and physiological circumstances of the host's flora, the ambient temperature, the humidity etc can act synergistic or antagonistic. Every intrinsically pathogenic species occurs in nature in numerous strains differing in virulence. The presence of virulence or the loss of virulence can be evaluated in test systems which are known to the skilled worker and is relevant for the respective animal poxvirus. The animal poxviruses of the present invention show in particular no virulence whatsoever in human hosts.

The term “pathogenicity” (pathos=suffering) refers to the property of an infectious pathogen or metazoic parasite of being able, after penetration, adhesion and identical replication in a host, to lead to a local or general impairment of the capacity to function (functio laesa) and to cause an infectious disease. Since the development of an infectious disease depends on the pathogen and host, the term pathogenicity relates to the pathogen-host system, not just to the pathogen. The pathogenicity relates to the species of a pathogen, not to a variant, a strain or a colony. It is a basic property, a power, which can, but need not, act. A pathogenic species cannot, relative to a particular pathogen-host system, become apathogenic in nature because this basic ability of a whole species is not lost.

The terms “immunogenicity” and “immunizing properties” are used synonymously in the present invention. Immunogenicity of an animal poxvirus refers to the ability of the animal poxvirus to induce in a vertebrate, preferably in the natural host of the virus or in a human, a cellular specific and/or humoral immune response, e.g. to stimulate T-cell proliferation and/or the generation of antibodies. The loss of the immunogenicity of the highly attenuated animal pox strains of the present invention is associated with the loss of this ability. The immunogenicity or the loss thereof can be investigated in test systems which are known to the skilled worker.

“Vector vaccines” (recombinant vaccines, hybrid vaccines) mean vaccines which consist of two components: a microbial carrier (vector) and an immunizing antigen whose coding nucleic acid is incorporated into the vector. Suitable and preferred microbial carriers are, because of their numerous nucleic acid deletions and their paramunizing properties, (highly) attenuated animal poxviruses. The introduced foreign gene nucleic acid is brought to expression by the vector in the vaccine, leading to the formation of specific immunizing reactions.

“Paramunity inducers” (paraspecific vaccines) refer to bioregulatory products composed of attenuated, avirulent and inactivated animal poxviruses which, depending on the degree of attenuation, now comprise only residues of (conventionally attenuated) or no (highly attenuated) immunizing properties and are intended to be used for paramunization in humans and animals. They are produced like conventional specific vaccines and also resemble them functionally, but with the difference that they activate predominantly the paraspecific (nonspecific) defense mechanisms and moreover lead, through their bioregulating properties, to homeodynamic defense systems.

### DETAILED DESCRIPTION OF THE INVENTION

### General

The invention is based on the surprising discovery that the virulence and immunizing properties of conventionally attenuated animal poxvirus strains can be reduced as far as complete loss by additional plaque terminal dilution passages in permissive cell cultures, incubated chicken eggs or experimental animals. Strains highly attenuated in this way are stable to a potential reactivation of these properties. This process, which goes beyond a simple, conventional attenuation, is referred to in the present invention as “high attenuation”. These highly attenuated animal poxvirus strains are distinctly improved over conventionally attenuated pathogens. In particular, the highly attenuated animal pox strains differ, as summarized in table 3, from the conventionally attenuated animal pox strains in gene technology terms through the decrease in the molecular weight of the viral nucleic acid and increase in deletions in the nucleic acid; in biological terms through the loss of virulence and contagiousness; in immunological terms through the loss of immunogenicity, and in molecular biology terms through the loss of cytokine receptors.

It was an unexpected discovery that all the tested attenuated animal pox strains of the family poxviridae, irrespective of the genus to which they belong, can be conventionally attenuated and then further weakened with high stability (see tables 1 and 2). Such a high attenuation is shown in this invention by way of example with representatives of the genera orthopoxviruses, leporipoxviruses and avipoxviruses, but it is not to be regarded as confined to these genera. The present invention also describes for the first time high attenuation with a myxomatosis virus and a camelpox virus.

The ability of infectious pathogens to change in order to adapt to environmental changes, e.g. by growing in cell cultures or in non-natural host systems, can be utilized experimentally in order to reduce markedly the time necessary for high attenuation. This preferably takes place by long-term passages in particular permissive host systems which do not normally belong to the natural host range (e.g. experimental animals, cell cultures, nutrient media). High attenuation of poxvirus strains ordinarily takes about 15 to 30 years.

Highly attenuated poxvirus strains also lose their specific immunizing capacities, whereas their paraspecific activity is specifically enhanced, by the highly attenuating method described in detail below. The highly attenuated poxvirus strains are therefore suitable as paramunity inducers or for producing vector vaccines.

The enhancement of the paraspecific properties is presumably attributable to mutual interference of immunizing and paramunizing proteins of animal poxviruses. The loss, caused by the high attenuation, of the immunizing proteins makes additional paramunizing proteins active, and they increase in a significant manner the paramunizing activity of these strains. In this way, highly active and harmless paramunity inducers are obtained and do not cause any allergies or other immunopathogenic side effects even if administrations are repeated in the short term and are frequent.

Ordinarily, simple, conventional attenuation leads to a reduction in virulence and contagiousness and to a limitation of the host range and to small changes in the pathogen genome with a simultaneous decrease in the molecular weight and the occurrence of deletions in the terminal regions of the viral genome. In addition there is a decrease in the specifically immunizing activities and an increase in the paraspecific activity. However, a high attenuation enhances these effects drastically, so that the resulting highly attenuated viruses are superior in terms of their stability, their host specificity, the lack of virulence and immunogenicity to conventionally attenuated viruses (tables 3 and 4).

### The Highly Attenuated Animal Poxviruses

In a first aspect, the present invention relates to a highly attenuated animal poxvirus based on an animal poxvirus strain of the family poxviridae, characterized in that the animal poxvirus no longer has any virulent and immunizing properties, and the highly attenuated animal poxvirus exhibits a lower molecular weight of the viral nucleic acid, more frequent deletions in the terminal region and an increased loss of cytokine receptors by comparison with conventionally attenuated animal pox strains. Known attenuated animal poxviruses have between 0 and 3 deletions in one or both terminal regions of the viral genome. The number of deletions in various strains of merely attenuated animal poxviruses varies, however, so that the highly attenuated animal poxviruses of the present invention taken together exhibit between 1, 2, 3, 4, 5 or more deletions in the terminal regions of the viral genome, than those in merely attenuated animal poxviruses. In a preferred embodiment of the invention, the highly attenuated animal poxviruses of the present invention exhibit in total 5, 6, 7, 8, 9, 10 or more deletions in the terminal regions. Exhibit preferably more frequent deletions, preferably at least 2 deletions in the right region and at least 2 deletions in the left region.

In a preferred embodiment of the highly attenuated animal poxvirus of the present invention, the viral genome shows a loss of cytokine receptors for interferon α and γ. It is particularly preferred for the animal poxvirus additionally to show also a loss of the receptors for IL-1 β and/or TH 1 cells.

In a preferred embodiment, the viral genome of the animal poxvirus is 16%, 17%, 18%, 19% and particularly preferably about 20% smaller than the viral genome of the wild type. The deletions are preferably located in one or both terminal regions of the animal poxvirus genome.

In a preferred embodiment, the highly attenuated animal poxvirus of the present invention is obtainable by the following method:

(a) adaptation of the animal pox in a permissive cell system or cell cultures, in particular lamb kidney cells or CAM cells;
(b) transfer and continuation of the animal poxviruses for attenuation by long-term passages in various permissive cell systems which make optimal infectious titers possible, e.g. in VERO cells, in particular for about 5 to 10 passages; in the case of myxomatosis virus; preferably at least 100, preferably at least 110, at least 120, at least 130 or more VERO cell passages are carried out, followed by at least 20, preferably at least 24 intermediate passages in AVIVER cell cultures and further VERO cell passages;
(c) transfer and continuation of the animal poxviruses for attenuation in an optimal cell system for about 100-300 passages, e.g. in MA-104 cells, VERO cells or AVIVER cells, in particular for 200 to 300 passages; and
(d) transfer and continuation of the animal poxviruses in VERO cells for at least 90 passages, preferably for at least 100, 110, 120, 130, 140, 150, 160, 170, 180, 190, 200, 210, 220, 230, 240, 250, 260, 270, 280, 290, 300 or more passages.

Plaque-purified passages are preferably carried out in one or more of steps (b), (c), and (d).

### The Highly Attenuating Method

A conventional attenuation (as well as the first steps of the high attenuation) starts with adaptation of isolated animal poxviruses in homologous or heterologous permissive cell systems such as, for example, cell cultures, incubated chicken eggs or in experimental animals. This is followed by attenuation by long-term passages in various permissive cell systems. The permissive cell systems appropriate for each viral strain are specifically selected for each species of animal poxvirus. The selection depends on the infectious titer of the viruses in the particular cell system. Moreover, the cell system selected for the passaging will yield the highest infectious titer for the particular species of virus. Such a cell system, in particular cell line, can be determined by the skilled worker by methods known in the prior art. This also corresponds at the same time to the optimal virus titer. The attenuation is continued by continuing the animal poxviruses for about 100-300, in particular 110, 120, 130, 140, 150, 160, 170, 180, 190, 200, 210, 220, 230, 240, 250, 260, 270, 280, 290 or 300 passages in these optimal cell systems. This is followed by a terminal phase characterized by 3-5 plaque terminal dilution passages. This material can be further processed in accordance with the further use.

All the representatives described herein of orthopox-, leporipox-, parapox- and avipoxviruses can be attenuated in a conventional way. The subsequent high attenuation took place by continuing the passages of the simply, conventionally attenuated virus strain in homologous or heterologous permissive host systems. The choice of the host system in turn depends on the animal pox species and is selected according to the aspects mentioned above (infectious titer). High attenuation takes place for example by continuing the simply attenuated orthopoxviruses in VERO cells or the simply attenuated avipoxviruses in embryonic chicken embryo fibroblasts (FHE) cell cultures. Poxviruses (e.g. ectromelia virus, camelpox virus) are preferably highly attenuated by at least 60 to 300 passages, depending on the particular virus strain, in VERO cell cultures (for example 150 or 260 passages). The leporipoxvirus myxomatosis strain is highly attenuated by about at least an additional 150 to 300 passages in MA and VERO cell cultures, preferably 290 passages. The avipoxvirus gallinae strain is highly attenuated by about 100 to 150 additional passages, preferably by 98 passages, in FHE cell cultures. The parapoxvirus strain is highly attenuated by a further 100 to 160, preferably by 164 passages (tables 3 and 4). It is preferred to use the so-called plaque terminal dilution method for passaging the virus strains (i.e. in the transfer and inoculation).

In general, primary chicken embryo fibroblast cultures (FHE) are used for highly attenuating the genus avipoxvirus, and permanent MA-104 monkey kidney cells (for short: MA cells) or VERO cells passages (ATCC CCL-81, WHO, American Type Culture Collection) and many others are used for all other genera such as orthopoxvirus, leporipoxvirus and parapoxvirus. A fully synthetic medium is preferably employed for culturing the MA or VERO cell cultures, with particular preference for the MEM medium (“minimal essential medium”) which comprises 5% to 20%, preferably 10% BMS (serum substitute medium) and 5% to 20%, preferably 10% lactalalbumin hydrolysate. After exchange with the culture medium, the virus medium preferably used is MEM medium with 5% to 20%, preferably with 10% lactalalbumin hydrolysate, without BMS and without fetal calf serum and without antibiotics. All the production methods are preferably carried out at pH values of from 7.0 to 8.0, preferably at a pH value of 7.25. Virus harvests with titer of 10⁵ to 10⁸ TCID₅₀/ml, preferably of at least 10^{7.5} TCID₅₀/ml, are preferred as starting material for producing the highly attenuated animal pox strains.

Replication of the poxviruses in VERO cells leads to a typical cytopathic effect which leads to destruction of the infected cells (lysis). With an initial inoculation dose of about 10 MOI (“multiplicity of infection”), a brief rounding phase (1-2 days) is followed by reticulated cell structures for about 3 days and by lysis of the cells after about 5 days.

The virus harvests obtained from the last passaging can be further processed appropriately for their use. For example, the nucleic acids present in the viruses can be cloned recombinantly to produce vector vaccines. Or the highly attenuated virus harvests can be lyophilized and be stored for example by adding 2.5% gelatin at 4° C. for further use, for example as paramunity inducers. For medical and therapeutic indications, the lyophilizate can be checked for its harmlessness and activity.

### Highly Attenuated Orthopoxviruses

In a preferred embodiment, the following highly attenuating method described by way of example with camelpox viruses can be used for orthopoxviruses:

Orthopoxvirus cameli, h-M 27

Camelpox viruses, isolated from pustular material from diseased animals, such as the strain M 27, are cultured in embryonic lamb kidney cell cultures for about 2 passages. The animal poxviruses cultured in this way are transferred by a suitable method, preferably by the plaque terminal dilution method, into VERO cells and continued there for about 5 passages. After passaging in a VERO cell culture, the last cell culture passage is adapted to MA cells (MA-104 monkey kidney cells) and continued for about 114 passages. The 121st plaque-purified MA passage obtained in this way (total of 284 passages) has proved to be simply attenuated. It is possible in isolates of this passage, i.e. with a simple attenuation, already to observe a decline in the virulence for the homologous host, a restriction of the host range, an increase in the infectious titer, a decrease in the giant cells in the cytopathic effect in cellcultures and a small decrease in the specific immunogenic activities. Because of the immunizing properties, which are still present after a simple attenuation, of the animal poxviruses, simply attenuated camelpox viruses are also suitable for parenteral vaccination against human variola or as vaccine against camelpox (O.-R. Kaaden, A. Walz, C. P. Czerny and U. Wernery, 1992: “Progress in the development of a camelpox vaccine”, Proc. 1th Int. Camel Conf., 1, 47-49).

The camelpox virus strain M 27 can be highly attenuated by continuing the attenuated strain in VERO cells. For this purpose it is necessary to carry out at least a further 50-150 passages, preferably 100 plaque-purified VERO passages. A highly attenuated camelpox virus h-M 27 (h=highly attenuated) obtained in this way proves to be extremely stable. Overall, therefore, about 384 cell culture passages are necessary to produce the highly attenuated camelpox virus of the invention. The exact number of passages is, however, not intended to be regarded as restrictive in this connection. The skilled worker will appreciate that modifications of the methods described herein and of the parameters used, especially of the number of cell passages or the cell line for highly attenuating an animal poxvirus strain, are within the scope of the invention.

The highly attenuated camelpox virus, strain h-M 27, obtained by the method of the invention exhibits a total loss of virulence and contagiousness for the homologous host and a high infectious titer in VERO cells (10^{7.25} CID₅₀/ml). It is therefore particularly suitable for use as paraspecific vaccine (paramunity inducer). It is moreover possible for the paramunity inducer based on highly attenuated animal poxviruses to be used both in a form capable of replication and in inactivated form. In inactivated form, the highly attenuated virus is treated as described below with beta-propiolactone (V. Fachinger, T. Schlapp, W. Strube, N. Schmeer and A. Saalmuller, 2000: “Pox-virus-induced immunostimulating effects on porcine leukocytes”, J. Virology 74, 7943-7951; R. Föster, G. Wolf and A. Mayr, 1994: “Highly attenuated poxvirus induce functional priming of neutrophils in vitro”, Arch. Virol. 136, 219-226; Mayr A., 1999: “Paraspezifischen Vaccinen aus Pockenviren (Paramunitätsinducer): “Eine neue Art von Impfstoff”, Ärztezschr. Naturheilverf. 40, 550-557; Mayr, A. 2000: “Paraspezifische Vaccine—Eine neue Art von Impfstoffen zur Regulation von Dysfunktionen in verschiedenen Korpersystemen”, Erfahrungsheilkunde (EHK) 49, 591-598).

With a simple attenuation, the length of the virus genome is already markedly reduced through the occurrence of deletions. The length of the initial virus genome (wild type) is about 193 900 bp, while the length of the genome of the attenuated M 27 strain is about 172 400 bp. The conventional attenuation thus results in a marked loss of nucleotides in the DNA. Restriction digestion with the restriction enzyme HindIII shows that in the genome four restriction fragments fewer are present in the analysis gel (Otterbein C. K., 1994, Vet. Med. Diss. Munich). In this case there are two deletions in the right and two deletions in the left terminal segment of the viral genome. The central, conserved region of the viral genome remains unchanged (C. Gubser, S. Hue, P. Kellam and G. L. Smith, 2004: “Poxvirus genomes: a phylogenetic analysis”, J. Gen. Virol. 85, 105-117). The length of the viral genome was further shortened by the high attenuation from 172 400 bp (attenuated virus) to 160 300 bp. The number of deletions rose from 4 to 5 (two in the left and 3 in the right terminal segment of the genome), with the central, conserved region of the viral genome remaining stable. The high attenuation further led to a loss of interferon α and γ receptors and further interleukin receptors and, surprisingly, also to activation of hematopoietic stem cells.

### Leporipoxvirus Myxomatosis, Myxomatosis Virus h-M 2

In a further embodiment of the invention, the high attenuation was carried out with the myxomatosis virus strain M 2. Once again, there was passaging in CAM cells, followed by several passages of VERO and AVIVER cells and finally further passages in VERO cells.

In a preferred embodiment, the method for producing paramunity inducers from highly attenuated myxoma viruses includes the steps:
(a) isolation from diseased animals via the chorioallantoic membrane of 10-day old chicken embryos
(CAM) and continuation in this system for at least 2 or more, e.g. 3, 4, 5, 6, 7, 8, 9, 10 or more passages;
(b) transfer and continuation of the isolated animal poxviruses for at least 120 or more, e.g. 125, 130, 135, 140, 145, 150, 155, 160, 165, 170, 175, 180 or more passages in VERO cell cultures;
(c) transfer and continuation of the animal poxviruses in AVIVER cells for at least 24 or more, e.g. 25, 30, 35, 40, 45, 50, 55, 60 or more passages;
(d) transfer and continuation of the animal poxviruses in VERO cells for at least 157 to 200 passages;
e) transfer and continuation of the animal poxviruses in MA cells for at least 114 to 150 passages;
f) transfer and continuation of the animal poxviruses in VERO cells for at least 179 passages.

In a further embodiment, the myxomatosis virus used for the attenuation from the edematous subcutis (left ear) of a European wild rabbit (genus Oryctolagus) suffering in a typical way from myxomatosis the myxoma virus was isolated by culturing on the chorioallantoic membrane (CAM) of chicken eggs (VALO eggs) incubated for 10 days, and adapted three times on the CAM in passages by the method of Herrlich A., Mayr A. and Munz E.: “Die Pocken”, 2nd edition, Georg Thieme Verlag, Stuttgart, 1967). The third CAM passage was adapted in a first stage to VERO cells over 120 passages (ATCC CCL-81, WHO, American Type Culture Collection), replicated in a 2nd stage by 24 intermediate passages in AVIVER cell cultures, and further cultured in the 3rd phase in VERO cells. In total, about 300 passages were carried out with the aim of attenuation. After these continuous terminal dilution passages, the originally virulent myxoma virus was attenuated.

The highly attenuated myxoma virus strain M 2 is obtained by continuing the attenuated strain in VERO cells. For this purpose it is necessary to carry out at least a further 250 to 350 passages, preferably 300 plaque-purified passages in VERO cells. The myxoma virus strain h-M 2 (h=highly attenuated) obtained in this way proved, like the highly attenuated camelpox strain, to be extremely stable. It likewise exhibits a total loss of virulence and contagiousness for the homologous host, a high infectious titer (10^{6.75} CID₅₀), complete loss of immunogenicity, increase in the paramunizing activity, further restriction of the host range, further deletions in the genome and loss of various interferon and interleukin receptors.

### Properties of the Highly Attenuated Orthopoxviruses

The highly attenuated animal pox strains of the invention are characterized as follows:
1. increased biological stability;
2. loss of virulence and contagiousness, even for 2-3-day old baby mice (parenteral, intraperitoneal);
3. loss of specific immunogenicity after parenteral and intraperitoneal administration;
4. complete restriction of the host range;
5. increase in the infectious titer of the attenuated virus in VERO cells;
6. strong paramunizing activity (capable of replication and inactivated);
7. shortening of the genome length of the attenuated animal poxviruses; corresponding decrease in the molecular weight;
8. increase in the number of deletions in the terminal region;
9. loss of the interferon α and γ receptor and other interleukin receptors;
10. activation of hematopoietic stem cells.

The number of cell passages and the types of cells necessary for conventional attenuation compared with high attenuation are compiled in table 3. Ordinarily, more than 100 to about 300 passages in various permissive host systems are necessary for high attenuation. A period of about 15-30 years is necessary for the complete attenuation.

Table 3 and table 4 show an overview of the biological and gene technological differences between a conventional attenuation and a high attenuation of the invention for the example of vacciniavirus, strain MVA. Thus, deletions frequently occur in the terminal regions of the viral genome (inverted terminal repeat) and the molecular weight is reduced owing to fewer base pairs. In the case of highly attenuated animal poxviruses, about 20% of the original genome is missing (which also therefore makes them so attractive as vector vaccines, see below). There is also found to be a loss of receptors, e.g. for IL-1β and TH 1 cells, and an enhancement of NK cell activation and of the formation of hematopoietic stem cells, and a further restriction of the host range in cell cultures. There is furthermore enhancement of interferon α and γ, IL-1, 2, 6, 12, and GM-CSA, TNF. Finally, the highly attenuated animal pox strains have no specific immunogenicity, but do have an increased activity of the nonspecific immune system (paramunity). There is a complete absence of virulence for humans or animals.

### Further Processing of Highly Attenuated Parapoxviruses to Paramunity Inducers

In the production of paramunity inducers from highly attenuated poxvirus strains it is possible to carry out an inactivation by chemical treatment with beta-propio-lactone at a concentration of 0.01%-1% beta-propiolactone. A concentration of 0.05% beta-propio-lactone is particularly preferred in this connection. Ideally, inactivation with beta-propiolactone is carried out at a pH of 7.8 and at 4° C. for about 1 hour while stirring and subsequently incubating at 37° C. for about 4 hours and overnight at +4° C. Inactivation with beta-propiolactone leads to a complete loss of the immunizing properties with a simultaneous large rise in the paraspecific activity.

In the production of paramunity inducers, the highly attenuated virus particles are preferably purified by centrifugation at low revolutions (e.g. 1000 rpm). After the centrifugation it is possible to add 0.5-10% succinylated gelatin (e.g. polygeline, obtainable from, for example, Hausmann, St. Gallen, Switzerland), preferably 5% succinylated gelatin. The resulting mixture can then be lyophilized in portions of for example 1.5 ml in appropriate sterile glass vials or ampoules and be dissolved with distilled water as required. A volume of 0.5-2 ml, preferably of 1.0 ml, of the lyophilizate dissolved in distilled water corresponds to a vaccine dose for humans on intra-muscular administration (see also Mayr A. and Mayr B.: “Von der Empirie zur Wissenschaft”, Tierarztl. Umschau, edition 57: 583-587, 2002). The lyophilized product can be stored stably for an unlimited time at temperatures of about +4° C. to +8° C. or at lower temperatures (e.g. −60° C.).

### Use of Highly Attenuated Animal Poxviruses as Paramunity Inducers

A further aspect of the invention relates to the use of highly attenuated animal poxvirus strains or of constituents of highly attenuated animal pox strains singly or combinations as paramunity inducers. Examples are freshly isolated animal poxviruses which are capable of replication or inactivated, recombinant animal poxviruses which are capable of replication or inactivated and which are derived from freshly isolated animal poxviruses, virus envelopes, detached envelopes, and cleavage products and aberrant forms of these envelopes, single native or recombinant polypeptides or proteins, especially membrane and surface receptors which occur in isolated animal poxviruses or are expressed recombinantly by a genetically modified poxvirus or a part of its genetic information.

A further aspect of the present invention is therefore to combine various highly attenuated pox strains of the same or another genus for use as paramunity inducers.

Because of their optimal paramunizing properties, the highly attenuated animal poxviruses are suitable for the following prophylactic or therapeutic indications in humans and animals:
multifactorial infectious factor diseases and mixed infections, chronic manifestations of infectious processes, obstinately recurrent infections, and chemotherapy-resistant, bacterial and viral infections;
defense weaknesses and dysregulations in the defense system of an organism;
neonatal threat of infection;
adjuvant therapy for certain neoplastic diseases, e.g. prevention of metastasis, reduction in side effects of chemo- and radiotherapy;
improvement in wound healing, avoidance of secondary infections following surgical procedures or through injuries;
regulation of the homeostasis between the hormonal, circulatory, metabolic, vascular and nervous systems.

The highly attenuated animal poxviruses, through the immediate onset of the paramunizing effect, promote harmlessness in relation to pathogens, thus neutralizing stress symptoms, latent infections, fever, a reduced general condition and other factors which may afflict immunization.

The paramunity inducers of the invention based on highly attenuated animal pox strains are thus suitable for inducing the paraspecific immune system and/or for the prophylaxis or treatment of deficiencies or multicausal infectious diseases. Examples of such diseases are dysfunctions of the immune system, immuno-suppression, immunodeficiency disorders, dysfunctions of the homeostasis between the hormonal, circulatory, metabolic and nervous systems, neonatal threat of infection, neoplastic diseases, viral diseases, bacterial diseases, therapy-resistant infectious factor diseases, mixed viral and bacterial infections, chronic manifestations of infectious processes, liver diseases of various origin, chronic skin diseases, herpetic diseases, chronic hepatitis, influenzal infections, endotoxin damage, improvement in wound healing with prevention of secondary infections.

Administration of the highly attenuated animal pox strains described herein can take place locally or parenterally. Local administration of paramunity inducers specifically stimulates the paraspecific defense mechanisms in the mucous membranes and in the skin. However, there is also a certain systemic effect. On the other hand, paramunizations applied parenterally scarcely influence the local defense mechanisms in the skin and mucosa. Preference is given in this connection to a pharmaceutical composition which includes one or more of the highly attenuated animal pox strains of the invention and, where appropriate, a pharmaceutically acceptable carrier.

Examples of such a carrier or additives are poly-ethylene glycol, dextrose, sorbitol, mannitol, poly-vinylpyrrolidone, gelatin, magnesiumstearate, carboxyl-polymethylene, carboxylmethylcellulose, cellulose acetate phthalate or polyvinyl acetate.

### Use of Highly Attenuated Animal Poxviruses as Vector Vaccines

A further aspect of the invention relates to the use of the highly attenuated poxvirus strains for producing vector vaccines (review: Pastoret, P.-P. and Vanderplasschen, A., 2003). Compared with conventionally attenuated strains, highly attenuated animal poxvirus strains are even more suitable as vectors for producing vector vaccines because they have completely lost their immunizing properties through the high attenuation. Since the viruses are passaged on the basis of their infectious titer, the deletions are located in regions which are unnecessary for viral replication. Owing to the deletions occurring in the terminal regions of the viral genome being even larger by comparison with conventional attenuation, the highly attenuated animal poxviruses provide sufficient space for inserting a foreign nucleic acid (DNA) to be expressed, or a foreign immunogen.

The foreign nucleic acid may code for a peptide or protein which provides immunizing epitopes. However, the invention is not intended to be restricted to a particular peptide or protein. The skilled worker will appreciate that the foreign genes can be cloned according to their size into the appropriate deletion region of the virus. Expression of the introduced peptide or protein can be controlled by control elements such as a promoter and, if necessary, by enhancer elements. The incorporation of a foreign nucleic acid which codes for a peptide or protein may induce a strong, specific immunostimulating property against the peptide or protein. This can be utilized for example by cloning into the vector construct a viral nucleic acid sequence whose expression induces an immune response in the transfected host.

The cloning of recombinant animal poxviruses as vector vaccines takes place after the last plaque terminal dilution passage. For the cloning, the viral nucleic acid can be cleaved with suitable restriction endonucleases and be ligated to the foreign nucleic acid sequences by standard ligation methods.

Compared with conventional vaccines or vector vaccines, for which other microbial vectors are used, the vector vaccines of the invention have the advantage that they have no allergic effect and provide an optimally regulated immune system to the eluted specific antigen, which contributes to an optimal inoculation result. The vector vaccines of the invention are also free of local or systemic negative side effects. Since they utilize the immunological interval until immunity develops fully, they are suitable in particular for emergency inoculations (e.g. in the case of acute risk of infection, before unexpected journeys).

The observation that the inoculation result and the harmlessness of the vaccines can be considerably increased through excellent paraspecific activity on the part of the vector animal poxvirus, is novel and makes the strains attractive for producing vector vaccines. Vector vaccines based on highly attenuated animal pox strains are therefore superior in terms of their activity and harmlessness to conventional vector vaccines.

### LIST OF TABLES

Table 1: Members of the family Poxviridae
Table 2: Classification of orthopoxviruses (genus Orthopoxvirus, OPV)
Table 3: Differences between conventional attenuation and high attenuation
Table 4: Number of passages in a conventional attenuation compared with a high attenuation.
Table 5: Administration regimen for treatment with paramunity inducer
Table 6: Indications for paramunization with the highly attenuated myxomavirus h-M 2.

### EXAMPLES

The following examples are preferred embodiments and serve to explain the invention further, but the latter is not intended to be restricted thereto.

### Example 1

As starting material for producing myxoma paramunity inducers (h-PIND-Myxo), conventionally attenuated myxomavirus M 2 (3 CAM passages, 277 VERO passages, 24 AVIVER passages=304 passages) were further passaged for a further 114 MA passages and 179 VERO passages (total 597 passages) and thus highly attenuated (see table 4). The VERO virus harvests of the highly attenuated Leporipoxvirus myxomatosis h-M 2 have a titer of at least 10^{6.75} CID₅₀/ml.

The highly attenuated leporipoxviruses obtained in this way showed no virulent or immunizing properties at all and were further processed to the paramunity inducer in the following way:

The virus harvests were inactivated with beta-propiolactone 0.05% (pH 7.8, 1 hour at +4° C. (stirred)), stirred at a temperature of +37° C. at a pH of 7.8 for 4 hours (monitored until the pH had adjusted to pH 7.8 if necessary), incubated overnight (stationary at a temperature of +4° C. for about 12 hours) and then purified by low-speed centrifugation (15 min, approx. 4000 g). Polygeline (pH 7.8) was added to the inactivated virus material for a total gelatin concentration of 2.5%. The virus material prepared in this way was dispensed into sterile 1.5 ml vials and lyophilized. The lyophilizates were stored at a temperature of +4° C. Before use, the lyophilizates were dissolved in 1 ml of sterile distilled water for injection and administered by deep intramuscular injection.

The administration sequences and medical indications are listed in table 5. The myxoma paramunity inducer is suitable for example for supportive treatment of herpes zoster (mode 4) by paramunization. In this case, the treatment leads to healing of the pustules which are typical of the disease after 3-4 days. In the case of preinfluenzal infections, on use of the paramunity inducers of the invention there is observed to be a complete disappearance of the symptoms (fever, lassitude, headaches and pain in the limbs). In patients with wound injuries (e.g. after operations), an unusually rapid wound healing, without secondary infections, is observed with the h-PIND-myxo. In cases of stomatitis and lesions associated with a visit to the dentist, rubbing in the lyophilizate was followed by the disappearance of the aphthae and lesions after 1-2 hours.

### Example 2

Conventionally attenuated camelpox virus M 27 (see description section) was highly attenuated by a further 263 passages in VERO cells (total 384 passages). Virus harvests over 10 CID₅₀/ml served as starting material for producing paramunity inducers. To this end, the virus harvest was inactivated, centrifuged and lyophilized in an analogous manner to example 1. The mode of administrations as well as the indications are analogous to those of example 1. The viruses obtained showed no virulent or immunizing properties at all owing to the high attenuation.

### Example 3

Conventionally attenuated canarypox virus (Avipox serinae, KP1, 535th FHE passage) was highly attenuated by a further 67 passages in FHE (see table 4). The 602nd FHE passage served in an analogous manner to example 1 and 2 as highly attenuated canarypox virus for producing paramunity inducers. The viruses obtained showed no virulent or immunizing properties at all owing to the high attenuation.

### Example 4

Conventionally attenuated fowlpox virus (Avipoxvirus gallinum, HP1, 444th FHE passage) was highly attenuated by a further 98th FHE passages. After the 542nd FHE passage, the fowlpox virus HP1 proved to be highly attenuated and was used in an analogous manner to example 1 for producing paramunity inducer. The viruses obtained showed no virulent or immunizing properties at all owing to the high attenuation.

### Example 5

Paramunity inducers based on myxomavirus (OPV muris) and parapoxviruses were also produced in analogy to the methods described in the preceding examples.

### Example 6

Highly attenuated animal pox strains are used to produce vector vaccines. In the production of vector vaccines, particular attention must be paid to monitoring the pH after each individual production step. The pH should be about 7.8. The attenuation and high attenuation of the viruses used for producing vectors takes place as described in example 1. It is possible in this connection to use all conventional gene technology methods for inserting nucleic acid segments which code for specific antigens against which a specific inoculation reaction, i.e. immunity development, is to be achieved. The foreign gene is routinely incorporated by means of suitable restriction enzymes into the deleted nucleic acid regions which have been generated by the high attenuation in the animal pox strains of the invention. Standard restriction digestions and cloning techniques are used in this connection.

It is possible to use for isolating recombinant virus constructs any (selection) marker genes or selection cassettes, such as, for example, the β-galactosidase gene, which are under the control of suitable control sequences.

Examples of methods for producing such vectors from animal pox strains are described in WO 00/69455. The disclosure of this publication and the teaching contained therein on the production of vector vaccines is hereby expressly incorporated by reference. Likewise, all other publications cited herein are incorporated by reference.

### Tabular Survey

TABLE 1
Members of the family Poxviridae

| Genus | Species | | Infectious disease |
|---|---|---|---|
| Orthopoxvirus | Opv. | commune | Vaccinia |
| | | variola/alastrim | Variola major/minor |
| | | simiae 1 | (human) |
| | | bovis | Monkeypox |
| | | cameli | “Cowpox” (and |
| | | muris | similar) |
| | | | Camelpox |
| | | | Mousepox |
| | | | (ectromelia) |
| Avipoxvirus | Apv. | gallinae | Fowlpox |
| | | meleagris | Turkeypox |
| | | columbae | Pigeonpox |
| | | serinis | Canarypox |
| | | coturnica, | Wild bird pox |
| | | agapornicis u.a. | |
| Capripoxvirus | Cpv. | ovis | Sheeppox (original) |
| | | caprae | Goatpox (original) |
| | | bovis | Lumpy skin disease |
| | | | (cattle) |
| Leporipoxvirus | Lpv. | myxomatosis | Myxomatosis |
| | | fibromatosis | (rabbits) |
| | | sciuris, leporis | Fibroma (rabbits) |
| | | | Squirrel fibroma, |
| | | | hare fibroma |
| Suipoxvirus | Spv. | suis | Swinepox |
| Parapoxvirus | Ppv. | ovis | Ecthyma, ORF (sheep, |
| | | bovis 1 | goat) |
| | | bovis 2 | Papular stomatitis |
| | | cameli | (cattle) |
| | | | Udder pox, milker's |
| | | | nodules |
| | | | Camel ecthyma |
| | | | (unofficial) |
| Yatapoxvirus | Ypv. | simiae 2 | Yaba monkey tumor |
| | | tanae | Tanapox (human, |
| | | | monkeys) |
| Molluscipoxvirus | Mpv. | molluscae | Molluscum |
| | | | contagiosum (human) |

TABLE 2
Classification of orthopoxviruses (genus Orthopoxvirus,
OVP)
(updated from the “7th Report of the International
Committee of Taxonomy of Viruses”, 2000)

| Species | Susceptible hosts |
|---|---|
| Variolavirus (OPV Variola) | Human |
| Vacciniavirus (OPV commune) | All mammals |
| Coxpox virus (OPV bovis) | All mammals |
| Ectromelia virus (OPV muris) | Mouse, fox, mink |
| Camelpox virus (OPV cameli) | Camel (experimentally possibly |
| | monkeys, mice, rabbits), |
| | humans are not susceptible |
| Monkeypox virus (OPV simiae) | Monkeys, human |
| Unclassified species: | Raccoon |
| Raccoonpox virus, California | Vole |
| volepox virus, taterapox | Rodents, hare, frog |
| virus | |

Note:
according to this still incomplete nomenclature, the following, previously included species are deleted: OPV bubali (buffalo), OPV elephant (elephant), OPV equi (horse), OPV cuniculi (rabbit)

TABLE 3
Differences between conventionally attenuated and highly attenuated MVA strains
(MVA = Modified Vaccinia Virus Ankara)
MVA original (572nd passage in primary chicken embryo fibroblast cultures) and VERO-MVA
(further 182 passages in permanent VERO cell cultures (WHO-ATCC, CCL 81))

| | MVA original (FHE) | VERO-MVA |
|---|---|---|
| Marker | (conventional attenuation) | (highly attenuated) |
| Genetic markers | 3 deletions in the left and right | 3 deletions in the right and 4 deletions in the |
| | terminal region (inverted terminal | left terminal region |
| | repeat) | |
| | Number of base pairs from 208 to 178 Kb | Number of base pairs 172 Kb |
| | Molecular weight: loss of 15% of the | Loss of 20% of the original genome |
| | original genome | |
| | Loss of the interferon α and γ receptor | Additionally further loss of receptors, e.g. for |
| | | IL-1 β and TH 1 cells |
| Cellular markers | Activation of T-helper cells | Enhancement of the activation of non-antigen- |
| | (CD 4, CD 8, CD 25) | specific cytotoxic T lymphocytes |
| | Activation of NK cells and | Enhancement of the NK cell activation and of the |
| | hematopoietic stem cells | formation of hematopoietic stem cells |
| | Abortive replication in mammalian cells | Further restriction of the host range in cell |
| | (exception: BHK) | cultures |
| Cytokines | Interferon α and γ, IL-2, 6 & IL-12, | Interferon α and γ, IL-1, 2, 6, 12, and M-CSA, |
| | M-CSA | TNF enhanced |
| Virus titer | FHE: 10^{9.5} CID₅₀/ml | FHE: 10^{4.5} CID₅₀/ml |
| | VERO: 10^{4.0} CID₅₀/ml | VERO: 10^{9.5} CID₅₀/ml |
| Immune system | Reduction in the activity of the | No specific immunogenicity; increased activity of |
| | specific immune system | the nonspecific immune system (paramunity) |
| Virulence for humans | weak | absent |
| and animals | | |

TABLE 4
Examples of numbers of passages in various selected cell cultures in conventional attenuation
compared with high attenuation of various animal pox strains

| | Number of passages for | Number of passages for high | |
|---|---|---|---|
| Virus strain | conventional attenuation | attenuation | Remarks |
| Modified vaccinia virus | 572 FHE passages | Further 182 passages in VERO | Conventional |
| MVA | (8, 9, 10, 19) | cells; total 754 passages | attenuation |
| Avipoxvirus HP 1 | 444 FHE passages (15) | Further 98 passages in FHE; | (published) |
| | | total 542 passages | High attenuation |
| Canarypoxvirus KP-1 | 535 FHE passages (4) | Further 67 FHE passages, | Subject matter of the |
| | | total 602 passages | present invention |
| Parapoxvirus ORF-D 1701 | 135 passages in embryonic | Further 164 passages in VERO | |
| | lamb kidneys (ELN); 37 pass. | cells; total 385 passages | |
| | in embryonic bovine lungs | | |
| | (BEL); 49 pass. in MA cells; | | |
| | total 221 passages (14) | | |
| Orthopoxvirus cameli, M 27 | 2 ELN passages; 5 VERO | Further 263 VERO passages; | Subject |
| | passage; 114 MA passages; | total 384 passages (h-M 27) | matter |
| | total 121 passages | | of the |
| Orthopoxvirus muris, | 3 CAM passages; 250 FHE | Further 50 FHE passages; 104 | present invention |
| Ectromelia virus Mü 1 | passages, total 253 passages | MA pass.; 93 VERO passages; | |
| | | total 500 passages | |
| Myxomavirus M 2 | 3 CAM pass.; 24 AVIVER pass.; | Further 179 VERO passages; | |
| (myxomatosis) | 277 VERO pass.; 114 MA pass.; | total 597 passages (h-M 2) | |
| | total 418 passages | | |

TABLE 5
Administration regimen for treatment with paramunity
inducer

| Mode of administration | Administration procedure |
|---|---|
| Mode 1 | 2 injections at interval of |
| (Prophylaxis, short-term) | 24 hours, 1-3 days before |
| | exposure |
| Mode 2 | 2-3 injections at interval |
| Prophylaxis, long-term) | of 24 hours; “courses” at |
| | monthly intervals |
| Mode 3 | 2 injections at interval of |
| (Prophylaxis, adjuvant | 24 h; once a month for |
| treatment of tumors and | months or years, possibly |
| other chronic disorders) | also more frequently |
| Mode 4 | 1-2 injections/day for 3-5 |
| (Parenteral therapy of | days or until the symptoms |
| acute infections) | disappear; additionally 1 |
| | injection/day until |
| | recuperation |
| Mode 5 | Administer undissolved |
| Nasal/oral prophylaxis or | lyophilisate deep into the |
| therapy) | nose or cheek pouch - for |
| | disorders at least twice a |
| | day; |
| | - only for disorders of the |
| | mucous membranes - |
| | (prophylactic application |
| | as required) |
| Mode 6 | Dissolve inducer in a |
| (cutaneous administration) | little ointment (note pH); |
| | ointment mixture must be |
| | used always freshly |
| | prepared |
| | - only for chronic skin |
| | disorders |

TABLE 6
Indications for paramunization with the highly attenuated myxomavirus h-M 2 (b-PIND-MYXO)
(Case examples from the family)

| Indication | Example | Patient | Result |
|---|---|---|---|
| Acute | Herpes zoster (Mode 4) | R.E., male, 57 y | Healing of the pustules within 3-4 |
| infections | Incipient influenzal | W.E., male, 22 y | days, no post-zoster encephalitis if |
| | infection | W.E., male, 75 y | the period of rest is observed, |
| | 1 injection i.m. when the | B.S., female, 67 y | complete disappearance of symptoms |
| | first symptoms appear, bed | B.M., female, 65 y | (fever, lassitude, in some cases |
| | rest for 2-3 hours | H.E., female, 63 y | headaches and pain in limbs) |
| | | B.B., male, 62 y | |
| Replacement | Operations (supporting | W.E., male, 75 y | Unusually rapid wound healing; no |
| and adjuvant | wound healing) Mode 5 | (Knee OP. 3 weeks after | secondary infections (according to |
| therapy | | zoster) | comments of the treating physicians) |
| | | E.B., female, 45 y (uterus) | |
| | | B.M., female, 60 y. (ankle) | |
| ENT region | Stomatitis, spontaneous | A.M., male, 82 y | After rubbing in the lyophilisate |
| | Lesions after visit to | B.M., female, 66 y | Disappearance of the apathy and of |
| | dentist | R.H., female, 67 y | the lesions after 1-2 hours |
| | Mode 5 | B.M., female, 65 y | |

### LITERATURE LIST

1. Smith, G. L., 1994: Virus strategies for evasion of the host response to infection Trends in Microbiol 2, 81-88.
2. Mayr, A., H. Stickl, H. K. Müller, K. Danner and H. Singer, 1978: Der Pockenimpfstamm MVA. Zbl. Bakt. Hyg. L. Abt. Orig. B 167, 375-390
3. Mayr, A., 1999: Geschichtlicher Überblick über die Menschenpocken (Variola), die Eradikation von Variola und den attenuierten Pockenstamm MVA. Berl. Münch. Tierärztl Wschr. 112, 322-328.
4. Mayr, A., F. Hartwig, and I. Bayr, 1965: Entwicklung eines Impfstoffes gegen Kanarienpocken auf Basis eines attenuierten Kanaraienpockenkulturvirus. Zbl. Vet. Med. B 12, 41-49.
5. Mayr, A. and K. Malicki, 1966: Attenuierung von virulentem Hühnerpockenvirus in Zellkulturen und Eigenschaften des attenuierten Virus. Zbl. Vet. Med. B 13, 1-13
6. Mayr, A. and M. Büttner, 1990: Ecthyma (ORF) virus: In: Dinter, Z. and B. Morein (eds.): Virus infections of vertebrates. Vol. 3: Virus infections of ruminants. Elsevier Science Publishers B.V. Amsterdam
7. Munz, E., 1999: Pox and pox-like diseases in camels. Proc. 1^{st} Int. Camel Conf. 1, 43-46.
8. Mayr, A. and C. P. Czerny, 1990: Camelpox virus. In: Dinter, Z. and B. Morein (eds.): Virus infections of vertebrates. Vol. 3: Virus infections of ruminants. Elsevier Science Publishers B.V. Amsterdam.
9. Otterbein, C. K., 1994: Phäno- and genotypische Untersuchungen zweier Kamelpockenvirusisolate vor und nach Attenuierung durch Ze\ddot{n}kulturpassagen Vet. Med. Diss. München
10. Kaaden, O. R., A. Walz, C. P. Czerny and U. Wernery, 1992: Progress in the development of a camelpox vaccine. Proc. 1th Int. Carmel Conf. 1, 47-49.
11. Gubser, C., S. Hue, P. Kellam and G. L. Smith, 2004: Poxvirus genomes: a phylogenetic analysis. J. Gen. Virol. 85, 105-117.
12. Fachinger, V., T. Schlapp, W. Strube, N. Schmeer and A. Saalmüller, 2000: Pox-virus-induced immunostimulating effects on porcine leukocytes. J. Virology 74, 7943-7951.
13. Förster, R., G. Wolf, and A. Mayr, 1994: Highly attenuated poxvirus induce functional priming of neutrophils in vitro. Arch. Virol. 136, 219-226.
14. Mayr, A., 1999: Paraspezifischen Vaccinen aus Tierpockenviren (Paramunitätsinducer): Eine neue Art von Impfstoff. Ärztezschr. Naturheilverf. 40, 550-557.
15. Mayr, A., 2000: Paraspezifische Vaccine—Eine neue Art von Impfstoffen zur Regulation von Dysfunktionen in verschiedenen Körpersystemen. Erfahrungsheilkunde (EHK) 49, 591-598.
16. Mahnel, H. J. Holejsovsky, P. Bartak and C. P. Czerny, 1993: Kongenitale “Ektromelie” bei Pelztieren durch Orthopoxvirus muris.
17. Mahenl, H., 1985: Schutzimpfung gegen Mäusepocken. Tierärzti. Prax. 13, 403-407.
18. Rolle, M. and A. Mayr (Hrsg.), 2002: Medizinische Mikrobiologie, Infektions-und Seuchenlehre. 7. Aufl. Enke Verlag Stuttgart.
19. Mahnel, H. 1983: Attenuierung von Mäusepockenvirus. Zbl. Vet. Med. B. 30, 701-710.
20. Pastoret, P.-P. and Vanderplasschen, A., 2003: Comparative Immunology, Microbiology & Infectious Diseases 26 (2003), 343-355.

1. A highly attenuated animal poxvirus based on an animal poxvirus strain of the family poxviridae, characterized in that the highly attenuated animal poxvirus no longer has any virulent and immunizing properties, and the highly attenuated animal poxvirus has a lower molecular weight of viral nucleic acid, more frequent viral nucleic acid terminal region deletions, and an increased loss of viral cytokine receptor encoding genes by comparison with conventionally attenuated animal poxvirus strains.

2. The highly attenuated animal poxvirus as claimed in claim 1, where the highly attenuated animal poxvirus exhibits a loss of viral genes for interferon α and γ cytokine receptors.

3. The highly attenuated animal poxvirus as claimed in claim 1, where the viral genome of the highly attenuated animal poxvirus is about 20% smaller than the viral genome of the wild type.

4. The highly attenuated animal poxvirus as claimed in claim 1, where the highly attenuated poxvirus is a camelpox virus strain.

5. The highly attenuated animal poxvirus as claimed in claim 4, where the camelpox virus strain is strain h-M 27 deposited under number 05040602 with the ECACC (European Collection of Animal Cell Cultures.

6. The highly attenuated animal poxvirus as claimed in claim 1, where the highly attenuated poxvirus is a myxoma virus strain.

7. The highly attenuated animal poxvirus as claimed in claim 6, where the myxoma virus strain is strain h-M 2 deposited under number 05040601 with ECACC.

8. The highly attenuated animal poxvirus as claimed in claim 1, where the highly attenuated animal poxvirus is fowlpox strain h-HP1.

9. The highly attenuated animal poxvirus as claimed in claim 1, where the highly attenuated animal poxvirus is ectromelia strain h-Mü1.

10. The highly attenuated animal poxvirus as claimed in claim 1, where the highly attenuated animal poxvirus is obtained by the following method:
(a) adaptation of animal poxviruses to a permissive cell system or cell cultures;
(b) transfer and continuation of the animal poxviruses for attenuation by long-term passages in various permissive cell systems;
(c) transfer and continuation of the animal poxviruses for attenuation in an optimal cell system for about 100-300 passages; and
(d) transfer and continuation of the animal poxviruses in VERO cells for at least 90 passages.

11. (canceled)

12. A paramunity inducer, based on a highly attenuated animal poxvirus as claimed in claim 1.

13. A method for producing highly attenuated animal poxviruses, comprising:
(a) adaptation of animal poxviruses to a permissive cell system or cell cultures;
(b) transfer and continuation of the animal poxviruses for attenuation by long-term passages in permissive cell systems;
(c) transfer and continuation of the animal poxviruses for attenuation in an optimal cell system for about 100-300 passages; and
(d) transfer and continuation of the animal poxviruses in VERO cells for at least 90 passages;
wherein carrying out steps (a)-(d) produces highly attenuated animal poxviruses.

14. A method for producing highly attenuated myxoma viruses, comprising:
(a) isolation of myxoma viruses from diseased animals via chorioallantoic membrane of 10-day old chicken embryos (CAM) and continuation in this cell system for at least 2 passages;
(b) transfer and continuation of the isolated myxoma viruses for at least 300 passages in AVIVER and VERO cell cultures from chicken embryos incubated for 10 days (FHE);
(c) transfer and continuation of the myxoma viruses in MA104 monkey kidney cell (MA) cells for at least 100 passages; and
(d) transfer and continuation of the myxoma viruses in VERO cells for at least 170 passages;
wherein carrying out steps (a)-(d) produces highly attenuated myxoma viruses.

15. The method as claimed in claim 14, where the highly attenuated myxoma viruses have an infectious titer of about 10^{6.75} CID₅₀/ml.

16. The method as claimed in claim 14, where the myxoma viruses are myxoma virus strain h-M 2 deposited under number 05040601 with ECACC.

17. A method for producing highly attenuated camelpox viruses, comprising
(a) isolation of camelpox viruses from diseased animals by culturing via chorioallantoic membrane (CAM) of 10-day old chicken embryos and continuation of the isolated camelpox viruses for about 2 passages in the CAM;
(b) transfer and continuation of the camelpox viruses for about 120 passages in VERO cells;
(c) transfer and continuation of the camelpox viruses for about 24 passages in AVIVER cells;
(d) transfer and continuation of the camelpox viruses for about a further 157 passages in VERO cells;
e) transfer and continuation of the camelpox viruses for a further 114 passages in MA cells;
f) transfer and continuation of the camelpox viruses for a further 179 passages in VERO cells;
wherein carrying out steps (a)-(f) produces highly attenuated camelpox viruses.

18. The method as claimed in claim 17, where the highly attenuated camelpox viruses have an infectious titer of about 10⁷ CID₅₀/ml.

19. The method as claimed in claim 17, where the camelpox viruses are camelpox strain h-M 27 deposited under number 05040602 with ECACC.

20. A vector vaccine comprising:
(a) viral nucleic acid of the highly attenuated, animal poxvirus as claimed in claim 1 and
(b) a nucleic acid sequence encoding an immunizing peptide or protein inserted into the viral nucleic acid.

21. The vector vaccine as claimed in claim 20, where the viral nucleic acid and the nucleic acid sequence encoding of the immunizing peptide or protein are present in a plasmid.

22. A pharmaceutical composition comprising a pharmaceutically acceptable carrier and one or more paramunity inducers as claimed in either of claim 12.

23. A method for inducing paraimmunity, comprising administering to a mammal an amount effective to activate the paraspecific immune system of the pharmaceutical composition as claimed in claim 22, wherein the administering comprises local or parenteral administration.

24. (canceled)

25. A method for inducing paraimmunity, comprising administering to a mammal an amount effective to activate the paraspecific immune system of a paramunity inducer as claimed in claim 12.

26. The method of claim 25 for the prophylaxis and/or treatment of an immunodeficiency-associated disorder.

27. The method as claimed in claim 26, where the immunodeficiency-associated disorder is selected from the group consisting of dysfunctions of the immune system, immunosuppression, immunodeficiency disorders, dysfunctions of the homeodynamics between the hormonal, circulatory, metabolic and nervous systems, neonatal threat of infection, neoplastic diseases, viral diseases, bacterial diseases, therapy-resistant infectious factor diseases, mixed viral and bacterial infections, chronic manifestations of infectious processes, liver diseases of varying origin, chronic skin diseases, herpetic diseases, chronic hepatitis, influenzal infections, endotoxin damage.

28. The method as claimed in claim 26, where the method is used for assisting wound healing and/or preventing secondary infections following surgical procedures or injuries.

29. A method for inducing a paraspecific and a specific immune response, comprising administering to a mammal an amount effective to activate the paraspecific and specific immune systems of a vector vaccine as claimed in either of claims 20-21.

30. (canceled)
